(19) United States
(12) Patent Application Publication
(10) Pub. No.: US 2020/0054314 A1
(43) Pub. Date: Feb. 20, 2020

(54) INTERVERTEBRAL INFLATABLE DISTRACTORS EMPLOYING THECAL SAC RETRACTORS, AND RELATED SYSTEMS AND METHODS

(71) Applicant: DESIGN ENTERPRISES, LLC, Cornelius, NC (US)

(72) Inventor: Benito J. GARRIDO, Cornelius, NC (US)

(73) Assignee: DESIGN ENTERPRISES, LLC, Cornelius, NC (US)

(21) Appl. No.: 16/104,883

(22) Filed: Aug. 18, 2018

(51) Int. Cl. A61B 17/02; A61F 2/44; A61F 2/46; A61B 17/70

(52) U.S. Cl. CPC A61B 17/025; A61F 2/4455; A61B 2017/00557; A61B 17/7074; A61F 2/4611

(57) ABSTRACT

Intervertebral inflatable distractors employing thecal sac retractors, and related systems and methods are disclosed. Adjacent vertebrae are spaced apart or distracted to prepare for the installation of intervertebral cages during spine surgery. An intervertebral inflatable distractor may include a inflatable portion having first and second inflation modes. In the lower-volume first mode, the inflatable portion may be efficiently received within the intervertebral space, then the higher-volume second mode may be used to abut against vertebrae endplates to urge them apart and thereby provide space to receive the cages. A thecal sac retractor of the intervertebral inflatable distractor may also be used to abut against the thecal sac to provide space for larger cages. In this manner, an interbody cage may be efficiently installed between vertebrae while minimizing injury to the vertebrae and the thecal sac.

### BACKGROUND

### Field of the Disclosure

The technology of the disclosure relates to medical devices and related assemblies and methods for distracting an intervertebral disc space.

### Technical Background

A vertebral column of a patient extends from the skull to the pelvis to provide support, movement, and protection to the patient. The column includes vertebrae separated by fibrocartilage structures (intervertebral disks) for shock absorption. A vertebral canal formed by openings through the vertebrae serves as a protective enclosure for a spinal cord of the patient from which branching nerves extend therefrom. The branching nerves and the spinal cord enable communication between the brain and other patient body parts. The column also helps support the patient's body, wherein ligaments, muscles and tendons interconnect and enabling relative movement between adjacent vertebrae.

Intervention may be necessary when abnormalities occur at the column. Examples of abnormalities necessitating medical intervention include degenerative disc disease necessitating intervertebral disc space height restoration for both disc prep and subsequent cage placement during interbody reconstruction and arthrodesis. Collapsed disc spaces may result from degenerative conditions and can be challenging to reconstruct and restore their height. Often, significant rigidity and distraction limitations exist secondary to inferior bone quality and or distractive force application location. Also, nearby locations of the spinal cord, branching nerves and nearby blood vessels can present inconvenient obstacles to when intervention requires access of the disc spaces. New approaches are needed to restore disc space heights in situations where the patient has inferior bone quality and traditional mechanical methods of distracting adjacent vertebrae would be undesirable due to unacceptable high local stresses place on adjacent vertebrae.

### SUMMARY OF THE DETAILED DESCRIPTION

Embodiments disclosed herein include intervertebral inflatable distractors employing thecal sac retractors, and related systems and methods. Adjacent vertebrae are spaced apart or distracted to prepare for the installation of intervertebral cages during spine surgery. An intervertebral inflatable distractor may include an inflatable portion having first and second inflation modes. In the lower-volume first mode, the inflatable portion may be efficiently received within the intervertebral space, then the higher-volume second mode may be used to abut against vertebrae endplates to urge them apart and thereby provide space to receive the cages. A thecal sac retractor of the intervertebral inflatable distractor may also be used to abut against the thecal sac to provide space for larger cages. In this manner, an interbody cage may be efficiently installed between vertebrae while minimizing injury to the vertebrae and the thecal sac.

In one embodiment, an intervertebral inflatable distractor is disclosed. The intervertebral inflatable distractor includes an inflatable portion configured to be disposed between endplates of first and second vertebrae of a patient. The inflatable portion includes a probe body extending from the thecal sac retractor. The inflatable portion further includes an inflatable body attached to the probe body, wherein the inflatable body in the first inflation mode is configured to be inserted by the probe body into a disc space of a patient, and the inflatable body is configured to distract the first and second vertebrae when in a second inflation mode. a portion of the fluid disposed within the inflatable body is less in the first inflation mode than in the second inflation mode. In this manner, injuries from pedicle screw insertion may be avoided and patient long-term comfort improved.

In another embodiment, a method for moving apart first and second vertebrae with an intervertebral inflatable distractor is disclosed. The method includes inserting the inflatable portion of the intervertebral inflatable distractor in the first inflation mode between first and second endplates. The method also includes abutting the inflatable body of the inflatable portion against the first and second endplates, by urging fluid into the inflatable body with a pressure control unit. The method also includes applying a force against the first and second vertebrae to distract the first and the second vertebrae to a distance corresponding to the second inflation mode by using the control input to adjust a portion of the fluid in the inflatable body. In this manner, a surgeon may precisely and efficiently control a distance between the vertebrae while avoiding patient injury.

Additional features and advantages will be set forth in the detailed description which follows, and in part will be readily apparent to those skilled in the art from that description or recognized by practicing the embodiments as described herein, including the detailed description that follows, the claims, as well as the appended drawings.

It is to be understood that both the foregoing general description and the following detailed description present embodiments, and are intended to provide an overview or framework for understanding the nature and character of the disclosure. The accompanying drawings are included to provide a further understanding, and are incorporated into and constitute a part of this specification. The drawings illustrate various embodiments, and together with the description serve to explain the principles and operation of the concepts disclosed.

### BRIEF DESCRIPTION OF THE FIGURES

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIGS. 1A and 1B are a left side schematic view and a superior sectional view, respectively, of an exemplary intervertebral inflatable distractor received in a disc space between endplates of first and second vertebrae of a patient and urging endplates of first and second vertebrae of the patient apart while abutting against a thecal sac of the patient;

FIG. 2A is a top view of the exemplary intervertebral inflatable distractor of FIG. 1A, including an inflatable portion in a first inflation mode;

FIG. 2B is a sectional view of the pressure line of the intervertebral inflatable distractor of FIG. 2A;

FIGS. 3A-1 and 3A-2 are left side views of the intervertebral inflatable distractor of FIG. 2A in first and second inflation modes, respectively;

FIGS. 3B-1 and 3B-2 are sectional views of the inflatable portion of the intervertebral inflatable distractor of FIG. 2A illustrating an inner space in the first and second inflation modes, respectively;

FIG. 4 is a right lateral partial view of first and second vertebrae of a spinal column defining a first Kambin's working triangle as is known in the art, wherein the first and second vertebrae are adjacent;

FIG. 5 is a superior sectional view of the second vertebra of FIG. 3 illustrating the first Kambin's working triangle as is known in the art with the first vertebra hidden;

FIG. 6A is a schematic anterior view of the inflatable portion of the intervertebral inflatable distractor of FIG. 2A in the first inflation mode and being received in the disc space between the first and the second vertebrae of FIG. 4;

FIG. 6B is a schematic right lateral view of the first Kambin's working triangle associated with the first and the second vertebrae of FIG. 6A;

FIG. 7A is a schematic anterior view of the inflatable portion of the intervertebral inflatable distractor of FIG. 6A being placed in the second inflation mode and urging the first and second vertebrae to distract by moving them further apart;

FIG. 7B is a schematic right lateral view of a second Kambin's working triangle associated with the first and second vertebrae of FIG. 7A represented by solid lines superimposed on the first Kambin's working triangle of FIG. 6B represented in broken lines;

FIG. 8A is a schematic right lateral view of a thecal sac retractor of the intervertebral inflatable distractor of FIG. 7A being placed in abutment with a thecal sac disposed between the first and the second vertebrae;

FIG. 8B is a schematic right lateral view of a third Kambin's working triangle associated with the first and second vertebrae of FIG. 8A represented by solid lines superimposed on the second Kambin's working triangle of FIG. 7B represented in broken lines;

FIG. 9 is a superior sectional view of the second vertebra of FIG. 1B illustrating the thecal sac retractor of the intervertebral inflatable distractor of FIG. 6A being placed in abutment with the thecal sac disposed between the first and the second vertebrae, wherein the first vertebra is hidden;

FIG. 10 is a superior sectional view of the second vertebra of FIG. 1B illustrating an intervertebral cage being received between the first and the second vertebrae, wherein the first vertebra is hidden; and

FIG. 11 is a superior sectional view of the second vertebra of FIG. 1B illustrating the inflatable portion of the intervertebral inflatable distractor in the first inflation mode being removed from the intervertebral space, wherein the first vertebra is hidden; and

FIG. 12 is a flowchart of an exemplary method for distracting the first and second vertebrae with the intervertebral inflatable distractor of FIG. 1A to install the intervertebral cage.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Reference will now be made in detail to the embodiments, examples of which are illustrated in the accompanying drawings, in which some, but not all embodiments are shown. Indeed, the concepts may be embodied in many different forms and should not be construed as limiting herein; rather, these embodiments are provided so that this disclosure will satisfy applicable legal requirements. Whenever possible, like reference numbers will be used to refer to like components or parts.

Embodiments disclosed herein include intervertebral inflatable distractors employing thecal sac retractors, and related systems and methods. Adjacent vertebrae are spaced apart or distracted to prepare for the installation of invertebral cages during spine surgery. An intervertebral inflatable distractor may include an inflatable portion having first and second inflation modes. In the lower-volume first mode, the inflatable portion may be efficiently received within the intervertebral space, then the higher-volume second mode may be used to abut against vertebrae endplates to urge them apart and thereby provide space to receive the cages. A thecal sac retractor of the intervertebral inflatable distractor may also be used to abut against the thecal sac to provide space for larger cages. In this manner, an interbody cage may be efficiently installed between vertebrae while minimizing injury to the vertebrae and the thecal sac.

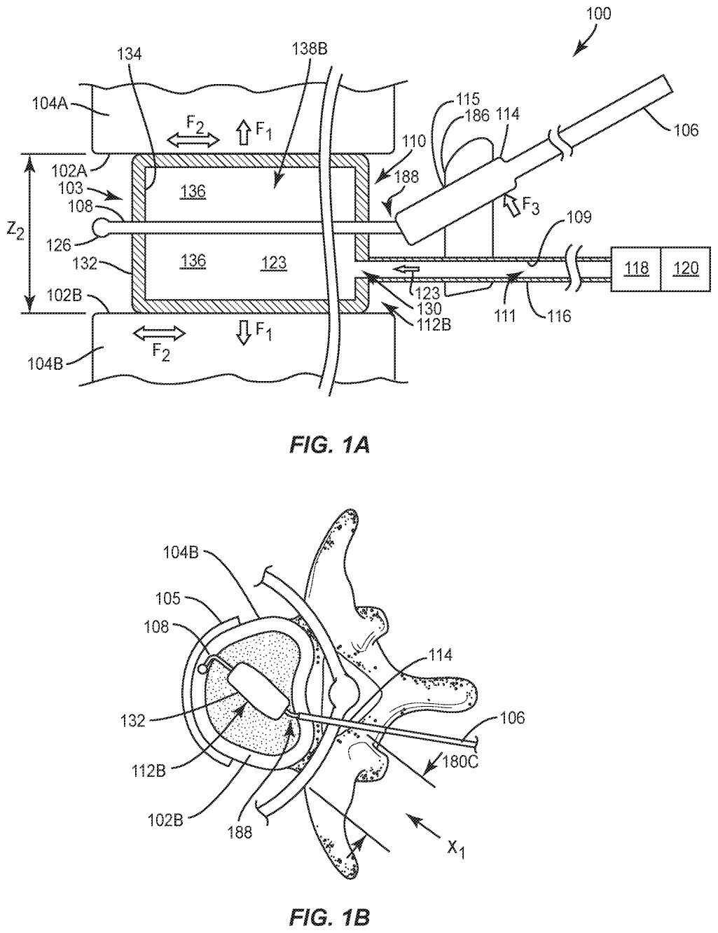

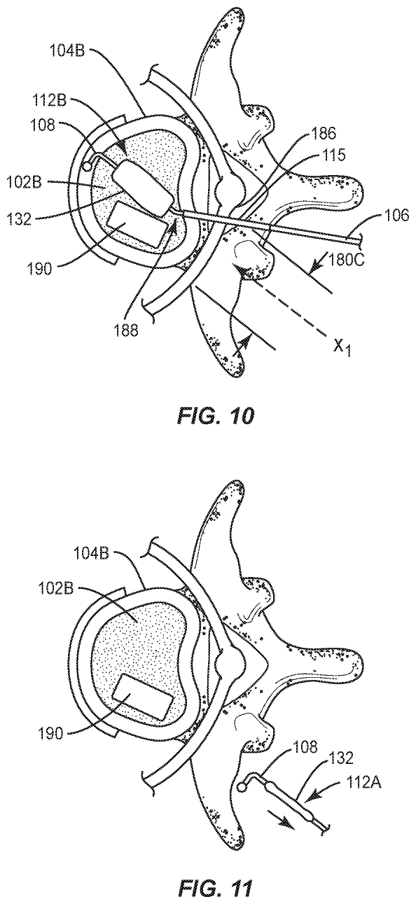

In this regard, FIGS. 1A and 1B are a left side schematic view and a superior sectional view, respectively, of an exemplary intervertebral inflatable distractor 100 received in a disc space 103 between endplates 102A, 102B of first and second vertebrae of a patient 101. The intervertebral inflatable distractor 100 may be used to urge apart the first and second vertebrae 104A, 104B with a force F1 to a height W3 during a medical intervention, for example, when an interbody cage 190 (FIG. 10) may be installed in the disc space 103.

An inflatable body 132 of the intervertebral inflatable distractor 100 is used to urge apart the endplates 102A, 102B in a technique often called “distraction.” In order to distract the endplates 102A, 102B, the inflatable body 132 is first inserted into the disc space 103. A handle 106 may be used by the attending surgeon to insert the inflatable body 132 into the disc space 103 through an opening in the patient 101. The handle 106 may be coupled the inflatable body 132 by a probe body 108. The probe body 108 may be flexible to enable the surgeon to explore the disc space 103 without damaging adjacent structures, for example blood vessels 107A, 107B just outside the endplates 102A, 102B. Once the surgeon has found a desired place for the inflatable body 132 within the disc space 132, then the inflatable body 132 may be pressurized with a fluid 123 using a pressure control unit 118 of the intervertebral inflatable distractor 100 which is in communication with an inner space 136 of the inflatable body 132 through a passageway 111 of the intervertebral inflatable distractor 100. The pressure control unit 118 may urge the fluid 123 through the passageway 111 and into the inner space 136 of the inflatable body 132. As the inflatable body 132 is filled with the fluid 123, a volume of the inflatable body 132 increases and the inflatable body 132 abuts against the endplates 102A, 102B with the force F1. The force F1 is applied to the endplates 102A, 102B by the inflatable body 132 and urges the endplates 102A, 102B apart. As a result of the abutment and the force F1 between the intervertebral inflatable distractor 100 and the endplates 102A, 102B, frictional forces F2 hold the intervertebral inflatable distractor 100 in place relative to the endplates 102A, 102A. These frictional forces F2 maintain the abutment as the handle 106 may be swung so that a support surface 115 of a thecal sac retractor 114 abuts against a thecal sac 186 of the patient 101 with a force F3. The force F3 displaces the thecal sac 186 and enables an access opening to the disc space 103 to enlarge to accommodate a larger interbody cage than could be otherwise inserted into the disc space if the thecal sac 186 were not displaced by the force F3.

Now that the basic operation of the intervertebral inflatable distractor 100 has been introduced, details of its components are now provided. In this regard, FIGS. 2A and 3A-1 are top and left side views, respectively, of an exemplary intervertebral inflatable distractor 100 in a first inflation mode 112A. The intervertebral inflatable distractor 100 may be received within the disc space 103 and distract the endplates 102A, 102B of adjacent first and second vertebrae 104A, 104B (FIG. 1A). The intervertebral inflatable distractor 100 includes the handle 106, an inflatable portion 110 configured for first and second inflation modes 112A, 112B, a thecal sac retractor 114, a pressure line 116, and the pressure control unit 118, and control input 120.

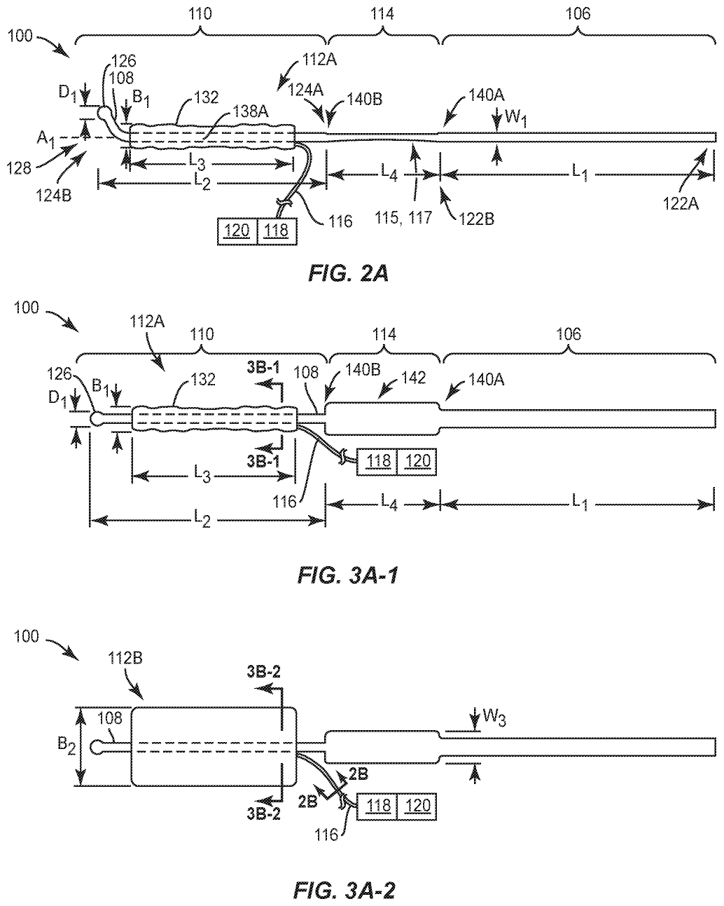

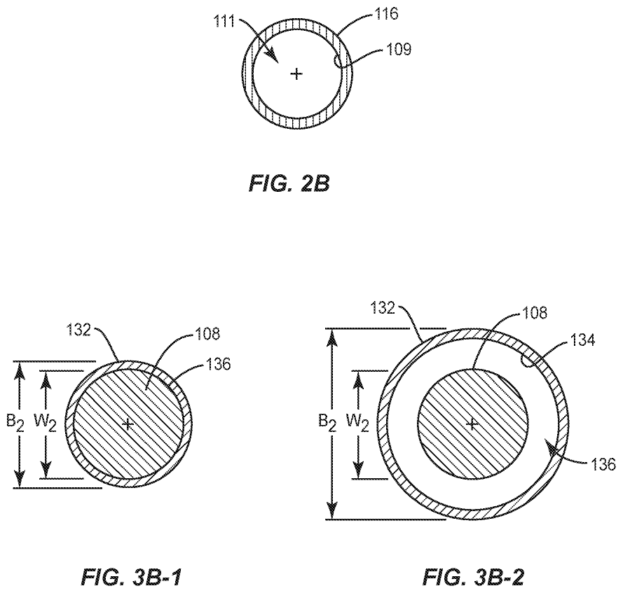

The pressure line 116 has an inner surface 109 forming a passageway 111 (FIG. 2B) from the pressure control unit 118 to a pressure port 130 at the inflatable body 132 of the inflatable portion 110. The pressure port 130 leads from the passageway 111 to the inner space 136 of an inflatable body 132 of the inflatable portion 110. The fluid 123 may pass through the passage 111 and the pressure port 130 to and from the inner space 136 of the inflatable body 132 to distract the first and second vertebrae 104A, 104B as the inflatable portion 110 transitions between the first and the second inflation modes 112A, 112B.

Details of each of the handle 106, the inflatable portion 110, the thecal sac retractor 114, the pressure line 116, the pressure control unit 118, and the control input 120 are now sequentially discussed.

First, the handle 106 may be used by an attending surgeon or robotic system (not shown) to locate the intervertebral inflatable distractor 100 relative to the first and the second vertebrae 104A, 104B. The handle 106 may extend from a first end 122A to a second end 122B a distance length L1 in a range from four to ten centimeters along a longitudinal axis A1 to fit in the hand of an attending surgeon. The handle 106 may also include an ergonometric-shape to be held or supported securely by the attending surgeon. The handle 106 may also include a width W1 in the range between 0.75 millimeters and two millimeters to minimize obstruction in the surgical environment. In this manner, the attending surgeon may position the intervertebral inflatable distractor 100 and operate the inflatable portion 110 as discussed later.

The inflatable portion 110 may enter the disc space 103 and distract the vertebrae 104A, 104B. The inflatable portion 110 includes the probe body 108 and the inflatable body 132 supported by the probe body 108. of the intervertebral inflatable distractor 100 relative to the handle 106. The probe body 108 is flexible to minimize opportunity for injury to the first and second vertebrae 104A, 104B or neighboring nerve fibers and yet provides enough firmness to hold the inflatable body 132 static relative to the handle 106 when the probe body 108 is free from abutment against the patient. The probe body 108 extends a length L2 in a range from 35 to 55 millimeters along the longitudinal axis A1 from a proximal end 124A to a distal end 124B, wherein the proximal end 124A is coupled to the first end 122A of the handle 106 through the thecal sac retractor 114. The probe body 108 may include a width W2 in a range from 15 to 30 millimeters.

The probe body 108 may also include a ball tip probe 126 having a diameter D1 in the range of one (1) to three (3) millimeters to further minimize opportunity for patient injury as the distal end 124B of the probe body 108 is received between the vertebrae 104A, 104B. The probe body 108 may include a bent portion 128 providing more flexibility to the ball tip probe 126 upon abutment against patient structures such as damage to the endplates 102A, 102B and an anterior longitudinal ligament 105 (FIG. 3). In this manner, the probe body 108 may reduce the opportunity for patient injury.

Next, the inflatable body 132 of the inflatable portion 110 facilitates the application of the force F1 to distract the endplates 102A, 102B of the first and the second vertebrae 104A, 104B in the context of the first and the second inflation modes 112A, 112B. With continued reference to FIG. 2A depicting the first inflation mode 112A, the inflatable portion 110 includes an inflatable body 132 connected to the probe body 108. The inflatable portion 110 extends a length L3 in a range from 25 to 35 millimeters, so that it may be consistent with the anterior-posterior width of endplates 102A, 102B of the first and second vertebrae 104A, 104B. In this manner, the inflatable body 132 may fit within the endplates 102A, 102B of the vertebrae 104A, 104B and later distribute the intervertebral distraction force F1 across the a significant portion of the width of the endplates 102A, 102B to avoid localized stresses that may cause patient injury.

Further, the inflatable body 132 of the inflatable portion 110 includes an inner surface 134 defining the inner space 136. This inner space 136 may contain a first portion 138A of the fluid 123 used to control a pressure within the inner space 136. The inner space 136 may be in fluid communication with the pressure control unit 118 through the pressure port 130. In this approach the pressure control unit 118 may maintain the first portion 138A of fluid 123 in the internal space and the volume of the inflatable body 132 so that a first outer width B1 is maintained as depicted in FIG. 2A and the first portion 138A may be zero as the inflatable body 132 may elastically shrink by default and expel much if not all of the first portion 138A of the fluid 123 through the pressure port 130 and into the pressure line 116. Given this tendency to elastically shrink, the first outer width B1 may be in a range from 10 to 25 millimeters in the first inflation mode 112A to enable the inflatable body 132 to be easily received within the disc space 103 between the first and the second vertebrae 104A, 104B.

The inflatable body 132 may also transition to the second inflation mode 112B to enable distraction. FIG. 3A-2 is a left side view of the intervertebral inflatable distractor 100 of FIG. 2A with the inflatable body 132 in the second inflation mode 112B. FIG. 3B-2 is a sectional view of the inflatable body 132 of FIG. 3A-2. The attending surgeon may increase the inflatable portion 110 from the first inflation mode 112A to the second inflation mode 112B using the control input 120 of the pressure control unit 118. In the second inflation mode 112B, a second portion 138B of the fluid 123 is contained in the inflatable body 132, and this increases the inner space 132 of the inflatable body 132 and an associated pressure therein, so that as the inflatable body 132 abuts against the endplates 102A, 102B of the first and second vertebrae 104A, 104B, the pressure with a force F1 distracts (or urges away) the endplates 102A, 102B from each other. As an inflatable body 132, the inner space may be at a constant pressure P2 resulting in the force F1 to be distributed along the length L3 of the inflatable body 132 that may be in abutment with the endplates 102A, 102B. This distribution of the force F1 can avoid localized stress that may cause patient injury. In this manner, the inflatable portion 110 enables distraction of the first and the second vertebrae 104A, 104B.

The inflatable body 132 facilitates the medical procedure conclusion, wherein the pressure control unit 118 may transition the inflatable body 132 back to the first inflation mode 112A. To perform this transition, the fluid 123 may depart from the inner space 136 via the pressure port 130 and back towards the pressure control unit 118. Once this fluid 123 has departed, then the intervertebral inflatable distractor 100 may return to the first inflation mode 112A as the pressure P2 within the inner space 136 is reduced to the pressure P1 causing the second outer width B2 of the inflatable body 132 in the second inflation mode 112B to be reduced to the first outer width B1 in the first inflation mode 112A. This first outer width B1 facilitates efficient removal of the inflatable body 132 from the disc space 103 with this smaller dimension and thereby has less likelihood causing patient injury during removal.

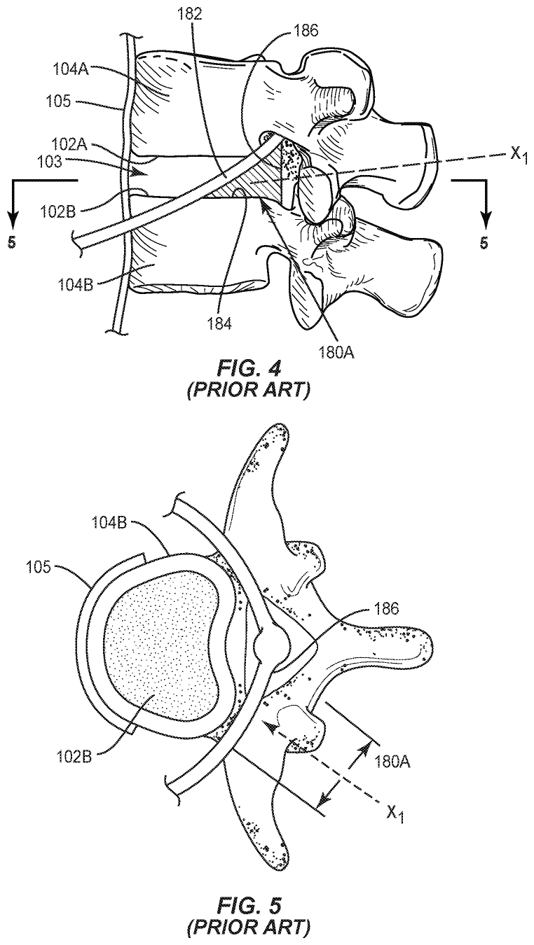

With continued reference to FIG. 2A, the thecal sac retractor 114 increases an opening of the pathway X1 (FIG. 4) so that better access to the disc space 103 can be achieved from the posterior direction. The thecal sac retractor 114 includes the support surface 115 configured to abut against the thecal sac 186 disposed between the vertebrae 104A, 104B. The support surface 115 may include a convex shape 117 to better surround and thereby better facilitate the secure abutment of the thecal sac 186. A secure abutment without slippage may be important because the thecal sac 186 is a membranous sheath surrounding the sensitive spinal cord of a patient. The thecal sac 186 is one of the patient structures that limits enlargement of the posterior access passageway to the disc space 103 of the vertebrae 104A, 104B. Accordingly, by temporarily laterally moving the thecal sac 186, a larger-sized opening of the posterior pathway X1 to the disc space 103 can be achieved.

The thecal sac retractor 114 includes features to facilitate temporary lateral movement of the thecal sac 186. The thecal sac retractor 114 may be coupled to the handle 106 and the probe body 108 of the inflatable portion 110. This close proximate arrangement to the handle 106 facilitates precise control of the thecal sac retractor 114 by the attending surgeon of the movement of the thecal sac 186 while the inflatable portion 110 remains in the second inflation mode 112B and the inflatable body 132 is secured in the disc space 103.

The thecal sac retractor 114 extends from a first side 140A to a second side 140B a distance L4. The distance L4 may be in a range from 15 millimeters to 25 millimeters. The thecal sac retractor 114 may also be a distance L2 from the ball tip probe 126. The distance L2 may be in a second range from 35 to 45 millimeters. The distances L2, L4 facilitate the thecal sac retractor 114 to be disposed proximate to the thecal sac 186 when distracting the endplates 102A, 102B.

The thecal sac retractor 114 includes a strong rigid material, for example, stainless steel, to facilitate firm application of an abutment force F3 to the thecal sac 186. The thecal sac retractor 114 includes an elongated width W3 in a range from 6 millimeters to 15 millimeters to distribute the abutment force F3 across the thecal sac 186 to reduce local stresses that may cause injury. The thecal sac retractor 114 may include a rectangular profile 142 to facilitate a larger abutment area than a circular cross section. In this manner, the thecal sac retractor 114 may temporarily move the thecal sac 186 while avoiding patient injury.

Next, with reference back to FIG. 2A, the pressure line 116 includes a passageway 111 providing a fluid connection between the inflatable body 132 and the control unit 118. The pressure line 116 may have an outer width W4 in a range from 3 millimeters to 8 millimeters to minimize obstruction in the surgical environment, as well as to provide sufficient size for the fluid 123 to communicate through the pressure line 116. The pressure line 116 may include a flexible material to provide convenient mechanical manipulation during surgery. In this manner, the pressure line 116 may provide fluid connectivity between the pressure control unit 118 and the inflatable body 132 in the surgical environment.

The pressure control unit 118 provides flow and pressure control of the fluid 123 into and out of the inflatable body 132 via the pressure port 130 and the passageway 111 of the pressure line 116. In one embodiment, the pressure control unit 118 may include a fluid pump to urge the fluid 123 into and out of the passageway 111. As the pressure control unit 118 provides additional amounts of the fluid 123 into the passageway 111, the resulting portion 138B of the fluid 123 may be contained in the inflatable body 132 to transition the intervertebral inflatable distractor 100 from the first inflation mode 112B to the second inflation mode 112B. The pressure control unit 118 may also operate in reverse to remove some of the fluid 123 form the passageway 111 causing the pressure to reduce in the inflatable body 132 and return to the first inflation mode 112A. In this manner, the pressure control unit 118 may transition the inflatable body 132 between the first and second inflation modes 112A, 112B to facilitate distraction of the endplates 102A, 102B.

The control input 120 may be used by the attending surgeon to provide operational instructions to the pressure control unit 118. The control input 120 may include, for example, mechanical, electrical, or electromechanical components. The control input 120 may provide operational instructions to the pressure control unit 118 to transition the inflatable body 132 between the first and second inflation modes 112A, 112B and to other inflation modes so that the distraction can be precisely controlled as to provide the desired distance Z2 between the endplates 102A, 102B necessitated for the medical procedure according to information received by the surgeon during the medical procedure. In this manner, the control input 120 may be used to guide the distraction of the endplates 102A, 102B.

To enable distraction, the intervertebral inflatable distractor 100 may be received in the disc space 103 via an approach which avoids patient injury in a minimally-invasive approach that may also reduce recovery time. In one embodiment, an exemplary approach may be to insert the intervertebral inflatable distractor 100 via a Kambin's working triangle which is formed by patient anatomic structures that demarcate a perimeter of a posterior patient opening by which the attending surgeon may safely access the disc space 103. In this regard, FIG. 4 is a right lateral partial view of the first and second vertebrae 104A, 104B of a spinal column defining a first Kambin's working triangle 180A as is known in the art. As illustrated in FIG. 4, the first Kambin's working triangle 180A is formed geometrically similar to a right triangle having a hypotenuse, base and height. Here, an exiting nerve 182 serves as the hypotenuse, superior-facing surfaces 184 of the second vertebra 104B serves as the base, and a thecal sac 186 serves as the height. The inflatable portion 110 in the first inflation mode 112 having the first outer width B1 fits efficiently through the first Kambin's working triangle 180A along a posterior pathway X1 through the first Kambin's working triangle 180A and into the disc space 103 where the distraction is desired. This pathway X1 is represented in FIG. 5 which is a superior sectional view of the second vertebra 104B of FIG. 4 illustrating the first Kambin's working triangle 180A as is known in the art, wherein the first vertebra 104A is hidden.

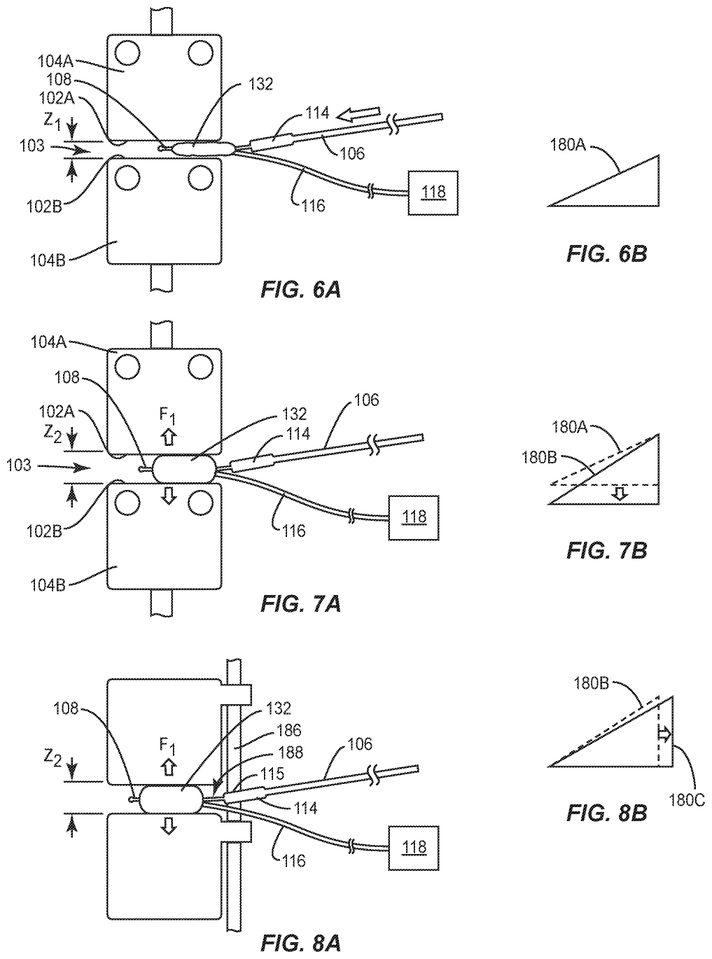

The inflatable portion 110 operation and the effect on the Kambin's working triangle is illustrated in FIGS. 6A through 8B. To begin, FIG. 6A is a schematic anterior view of the inflatable portion 110 of the intervertebral inflatable distractor 100 of FIGS. 2A and 2B in the first inflation mode 112A and being received in the disc space 103 between the first and the second vertebrae 104A, 104B of FIG. 4. The corresponding Kambin's triangle is shown in FIG. 6B which is a schematic right lateral view of the first Kambin's working triangle 180A associated with the first and second vertebrae 104A, 104B of FIG. 6A. In this manner, the intervertebral inflatable distractor 100 is inserted between the first and the second vertebrae 104A, 104B.

Distraction of the vertebrae 104A, 104B occurs as the inflatable portion 110 transitions to the second inflation 112B. In this regard, FIG. 7A is a schematic anterior view of the inflatable portion 110 of the intervertebral inflatable distractor 100 of FIG. 5A being placed in the second inflation mode 112B and urging the first and second vertebrae 104A, 104B to distract or otherwise move further apart. In this situation, the fluid 123 is urged by the pressure control unit 118 into the inner space 136 of the inflatable portion 110. As is discussed earlier, the inner space 136 is in fluid communication with the pressure control unit 118 through the passageway 111 of pressure line 116. As the fluid 123 is urged into the inner space 136 of the inflatable portion 110, the inflatable body 132 expands until it reaches the second inflation mode 112B as designated by the attending surgeon. During expansion, the inflatable body 132 abuts against the endplates 102A, 102B of the vertebrae 104A, 104B and thereby applies a force F1 that forces the endplates 102A, 102B further apart to a distance of Z2. FIG. 9 is a superior sectional view of the second vertebra 104B of FIG. 1A illustrating the inflatable portion 110 of the intervertebral inflatable distractor 100 of FIG. 6A being placed in the second inflation mode 112B and urging the first and the second vertebrae 104A, 104B to move further apart, wherein the first vertebra 104A is hidden. Note that the inflatable body 132 may contact only a portion of the endplate 102B and thereby provide space for the later insertion of an intervertebral cage that will also contact the endplates 102A, 102B. In this manner, intervertebral distraction may occur.

The first Kambin's working triangle 180A increases in size in response to the intervertebral distraction. FIG. 7B is a schematic right lateral view of a second Kambin's working triangle 180B associated with the first and second vertebrae 104A, 104B of FIG. 7A, wherein the second Kambin's working triangle 180B is represented by solid lines is superimposed on the first Kambin's working triangle 180A of FIG. 6B represented by broken lines. As is seen in this illustration, the Kambin's working triangle is enlarged as the intervertebral distraction increases the base of the Kambin's working triangle. The beneficial result of an enlarged Kambin's triangle is that a pathway X1 having a larger cross-section is created that can accommodate larger intervertebral cages.

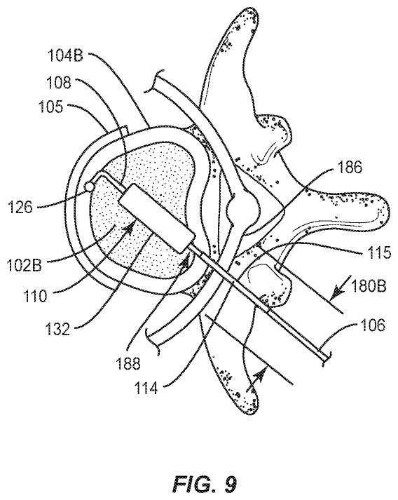

The second Kambin's working triangle 180B may be further enlarged to a third Kambin's working triangle 180C. FIG. 8A is a schematic right-side view of the thecal sac retractor 114 of the intervertebral inflatable distractor 100 of FIG. 7A being placed in abutment with the thecal sac 186 disposed between the first and second vertebrae 104A, 104B. The force F1 against the endplates 102A, 102B during the second inflation mode 112B facilitates frictional forces F2 to be created between the inflatable body 132 and the endplates 102A, 102B. These frictional forces F2 resist movement of the inflatable body 132 relative to the endplates 102A, 102B and create a pivot point 188 for the thecal sac retractor 114. The pivot point 188 enables the handle 106 to direct the support surface 115 of the thecal sac retractor 114 to abut against the thecal sac 186 while the inflatable body 132 remains anchored in the disc space 103 by the frictional forces F2. The pivot point 188 may include a flexure point (or hinge) of the probe body 108 disposed between the inflatable body 132 and the thecal sac retractor 114. The handle 106 may also enable the thecal sac retractor 114 to apply a force F3 against the thecal sac 186 and at least partially laterally distract the thecal sac 186 to increase a width of the second Kambin's working triangle 180B to create a third Kambin's working triangle 180C. In this regard, FIG. 8B is a schematic right lateral view of the third Kambin's working triangle 180C represented by solid lines superimposed on the second Kambin's working triangle 180B of FIG. 7B. The larger third Kambin's working triangle 180C enables an even larger intervertebral cage to be inserted through the passageway X1 than with the second Kambin's working triangle 180B.

Using the pivot point 188 also creates more free space for a subsequent insertion of the interbody cage 190 through the third Kambin's working triangle 180C. In this regard, FIG. 9 is a superior sectional view of the second vertebra 104B illustrating the thecal sac retractor 114 prior to abutment with the thecal sac 186. In contrast, FIG. 1A is the same view with the thecal sac retractor 114 in abutment with the thecal sac 186 resulting in the larger third Kambin's working triangle 180C. As illustrated in FIG. 1B, the handle 106 is moved out (or mostly out) of the third Kambin's working triangle 180C so that the handle 106 is much less of an obstruction for the passageway X1 formed by the third Kambin's working triangle 180C.

Due to the larger Kambin's working triangle, larger intervertebral cages may be received into the disc space 103. FIG. 10 is a superior sectional view of the second vertebra 104B of FIG. 9 illustrating the interbody cage 190 being received between the first and the second vertebrae 104A, 104B. In FIG. 10 the first vertebra 104A is hidden from view to show that the interbody cage 190 adjacent to the inflatable body 132 and unobstructed from the intervertebral inflatable distractor 100. In this manner, the interbody cage 190 may be more efficiently installed.

The intervertebral inflatable distractor 100 may also be efficiently removed. The pressure control unit 118 may be used by the attending surgeon to remove at least some of the fluid 123 in the inner space 136 of the inflatable body 132. This fluid 123 removal causes the inflatable portion 110 to return to the first inflation mode 112A which is evidenced by decreasing the volume of the inflatable body 132 as well as the force F1 of the inflatable body 132 against the endplates 102A, 102B which consequently reduces the frictional forces F2 between the inflatable body 132 against the endplates 102A, 102B. Once in the first inflation mode 112A, the intervertebral inflatable distractor 100 may be removed from the first and the second vertebrae 104A, 104B as is illustrated in FIG. 11 which is a superior sectional view of the second vertebra 104B of FIG. 10 wherein the intervertebral inflatable distractor 100 in the first inflation mode 112A being removed from the disc space 103 wherein the first vertebra 104A is hidden from view. In this way, the intervertebral inflatable distractor 100 may be removed from the disc space 103.

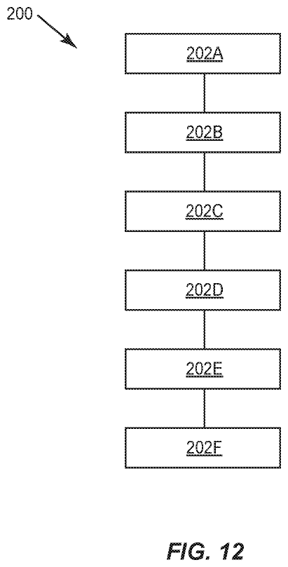

In summary, FIG. 12 is a flowchart of an exemplary method 200 for distracting the first and second vertebrae 104A, 104B with the intervertebral inflatable distractor 100 of FIG. 2A to enable an installation of the interbody cage 190. The method 200 is now discussed using the terminology discussed above in relation to the operations 202A through 202F as depicted in FIG. 12. The probe body 108 including inserting the inflatable portion 110 of the intervertebral inflatable distractor 100 (see FIG. 5A) in the first inflation mode 112A through the first Kambin's working triangle 180A and between the first and second endplates 102A, 102B (operation 202A of FIG. 12). The method 200 also includes abutting the inflatable body 132 against the first and second endplates 102A, 102B by urging the fluid 123 into the inflatable body 132 with the pressure control unit 118 (operation 202B of FIG. 12). The method also includes applying the force F1 against the first and second vertebrae 104A, 104B to distract the vertebrae to a distance Z2 corresponding to the second inflation mode 112B by using the control input 120 to adjust the amount of the fluid 123 in the inflatable body 132 (operation 202C of FIG. 12). The method 200 also may include distracting the thecal sac retractor 114 to abut against the thecal sac retractor 114 with a force F3 by moving the thecal sac retractor 114 about the pivot point 188 while the inflatable body 132 is secured in the disc space 103 by the frictional forces F2 against the thecal sac 186 (operation 202D of FIG. 12). The method 200 may also include positioning the interbody cage 190 between the first and the second vertebrae 104A, 104B as shown in FIG. 10 (operation 202E of FIG. 12). The method 200 may also include removing the portion of the fluid 123 from the inflatable body 132 to transition the inflatable body 132 to the first inflation mode 112A and removing the intervertebral inflatable distractor 100 from the first and the second vertebrae 104A, 104B as shown in FIG. 11 (operation 202F of FIG. 12).

Many modifications and other embodiments not set forth herein will come to mind to one skilled in the art to which the embodiments pertain having the benefit of the teachings presented in the foregoing descriptions and the associated drawings. Therefore, it is to be understood that the description and claims are not to be limited to the specific embodiments disclosed and that modifications and other embodiments are intended to be included within the scope of the appended claims. It is intended that the embodiments cover the modifications and variations of the embodiments provided they come within the scope of the appended claims and their equivalents. Although specific terms are employed herein, they are used in a generic and descriptive sense only and not for purposes of limitation.

What is claimed is:

1. An intervertebral inflatable distractor, comprising:
an inflatable portion configured to be disposed between endplates of first and second vertebrae of a patient, the inflatable portion including
a probe body extending from the thecal sac retractor, and
an inflatable body attached to the probe body, wherein the inflatable body in the first inflation mode is configured to be inserted by the probe body into a disc space of a patient, and the inflatable body is configured to distract the first and second vertebrae when in a second inflation mode,
wherein a portion of the fluid disposed within the inflatable body is less in the first inflation mode than in the second inflation mode.

2. The intervertebral inflatable distractor of claim 1, further comprising a handle coupled to the probe body, wherein the handle is configured to be held by a surgeon.

3. The intervertebral inflatable distractor of claim 2, further comprising a thecal sac retractor extending from the handle, the thecal sac retractor including a support surface configured to abut against a thecal sac disposed between first and second vertebrae;

4. The intervertebral inflatable distractor of claim 3, wherein the handle is configured to abut the thecal sac retractor against a thecal sac of the patient while the inflatable body is held secure in the disc space

5. The intervertebral inflatable distractor of claim 3, wherein the support surface includes a convex shape.

6. The intervertebral inflatable distractor of claim 3, wherein the thecal sac retractor includes an elongated width W3 in a range from 6 millimeters to 15 millimeters.

7. The intervertebral inflatable distractor of claim 1, wherein the inflatable body surrounds the probe body.

8. The intervertebral inflatable distractor of claim 1, wherein a distal end of the probe body includes a ball tip.

9. The intervertebral inflatable distractor of claim 1, wherein the probe body includes a flexible material.

10. The intervertebral inflatable distractor of claim 1, wherein the fluid is a liquid.

11. A method for moving apart first and second vertebrae with an intervertebral inflatable distractor, the method comprises:
inserting the inflatable portion of the intervertebral inflatable distractor in the first inflation mode between first and second endplates;
abutting the inflatable body of the inflatable portion against the first and second endplates, by urging fluid into the inflatable body with a pressure control unit; and
applying a force against the first and second vertebrae to distract the first and the second vertebrae to a distance corresponding to the second inflation mode by using the control input to adjust a portion of the fluid in the inflatable body.

12. The method of claim 11, wherein the inserting occurs through a first Kambin's working triangle.

13. The method of claim 11, further comprising receiving an intervertebral cage through a Kambin's working triangle and into the disc space.

14. The method of claim 11, further comprising positioning the intervertebral cage between the first and the second vertebrae.

15. The method of claim 11, further comprising distracting the thecal sac retractor to abut against the thecal sac retractor with a force by moving the thecal sac retractor about the pivot point while the inflatable body is secured in the disc space by the frictional forces against the thecal sac.

16. The method of claim 11, further comprising removing the portion of the fluid from the inflatable body to transition the inflatable body to the first inflation mode and removing the intervertebral inflatable distractor from the first and the second vertebrae
